(19) United States
(12) Patent Application Publication
(10) Pub. No.: US 2025/0334509 A1
(43) Pub. Date: Oct. 30, 2025

(54) POLARIMETRIC SWEAT-SENSING OF GLUCOSE

(71) Applicant: Brown University, Providence, RI (US)

(72) Inventors: Jingming XU, Dighton, MA (US); Seokkee MIN, Pawtucket, RI (US)

(21) Appl. No.: 19/189,108

(22) Filed: Apr. 24, 2025

Related U.S. Application Data
(60) Provisional application No. 63/638,869, filed on Apr. 25, 2024.

(51) Int. Cl. G01N 21/21; B01L 3/00; G01N 33/49

(52) U.S. Cl. CPC G01N 21/21; B01L 3/5088; G01N 33/49; B01L 2300/0654; B01L 2300/168; G01N 2201/0633

(57) ABSTRACT

A microfluidic chip includes a transparent or reflective substrate, a sample array of microfibers mounted on a first double-sided adhesive sheet adhered to the substrate such that the microfibers are facing up and away from the substrate, a shim added onto the first double-sided adhesive sheet adjacent to the sample array of microfibers such that a microfluidic channel is formed above the sample array of microfibers, a fluidic inlet added as a fill port to the microfluidic channel, and a second double-sided adhesive sheet laid over the shim to cover the microfluidic channel.

### CROSS REFERENCE TO RELATED APPLICATIONS

This application claims benefit from U.S. Provisional Patent Application Ser. No. 63/638,869, filed Apr. 25, 2024, which is incorporated by reference in its entirety.

### STATEMENT REGARDING FEDERALLY SPONSORED RESEARCH OR DEVELOPMENT

This invention was made with government support under grant number 80NSSC23M0162 awarded by the National Aeronautics and Space Administration, and grant number W911NF-21-1-0181 awarded by the US Army Research Office. The government has certain rights in the invention.

### BACKGROUND OF THE INVENTION

The present invention relates generally to sensing of glucose, and more particularly to polarimetric sweat-sensing of glucose.

In general, microfluidic technology has revolutionized the field of analytical chemistry and biological research by enabling the manipulation of small volumes of fluids in channels with dimensions of tens to hundreds of micrometers. This technology allows for the miniaturization of laboratory processes, leading to the development of lab-on-a-chip devices that can perform complex analyses with high precision and efficiency. The integration of microfluidic systems with optical components, such as polarimeters, has opened new avenues for the analysis of chemical and biological samples, providing insights into their optical properties and interactions.

The demand for advanced microfluidic devices is driven by the need for more efficient, accurate, and versatile analytical tools in various fields, including medical diagnostics, environmental monitoring, and chemical synthesis. Traditional methods of sample analysis often require large sample volumes and extensive preparation, which can be time-consuming and costly. Microfluidic devices, on the other hand, offer the advantage of reduced sample and reagent consumption, faster processing times, wearable form factors, and the potential for automation and integration with other analytical techniques. Despite these advantages, challenges remain in the design and fabrication of microfluidic devices.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In an aspect, the invention features a microfluidic chip including a transparent or reflective substrate, a sample array of microfibers mounted on a first double-sided adhesive sheet adhered to the substrate such that the microfibers are facing up and away from the substrate, a shim added onto the first double-sided adhesive sheet adjacent to the sample array of microfibers such that a microfluidic channel is formed above the sample array of microfibers, a fluidic inlet added as a fill port to the microfluidic channel, and a second double-sided adhesive sheet laid over the shim to cover the microfluidic channel.

In another aspect, the invention features an optical polarimeter including a light source, a polarizer, a microfluidic chip, an analyzer, and a detector.

In still another aspect, the invention features a method of fabricating a microfluidic chip including providing a microscope slide as a substrate to ensure local or microscopic flatness, providing a sample wire array, mounting the sample wire array on a first adhesive sheet, and placing the sample wire array on the first adhesive sheet on the microscope slide such that wires emanating from the sample wire array are facing up and away from the microscope slide.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE SEVERAL VIEWS OF THE DRAWING

FIG. 1 is a block diagram of an exemplary system.

FIG. 2 is a cross-sectional schematic of an exemplary microfluidic chip.

FIG. 3 is a flow diagram.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

Diabetes mellitus is a chronic disease due to a defect of insulin secretion and/or action, affecting a sizable portion of the world population. Blood Glucose (BG) concentration is an important biomarker used to track and diagnose diabetes. The most prevalent method of tracking BG is electrochemical sensing via glucose oxidase-coated electrodes. Many different optical BG sensors have been proposed, that assess blood, saliva, sweat, interstitial fluid, tear, various tissues, and urine. Detection of glucose in fluids other than blood, such as sweat, provide an effective non-invasive path to estimating BG due to their high correlation. Mechanisms of action of the optical sensors use various physical properties of light, such as polarimetry, colorimetry, coherence, scattering, and the luminescence of fluorescent materials. However, currently available BG sensors have issues, including, but not limited to, invasiveness, pain, intermittent measurements, and infection risk.

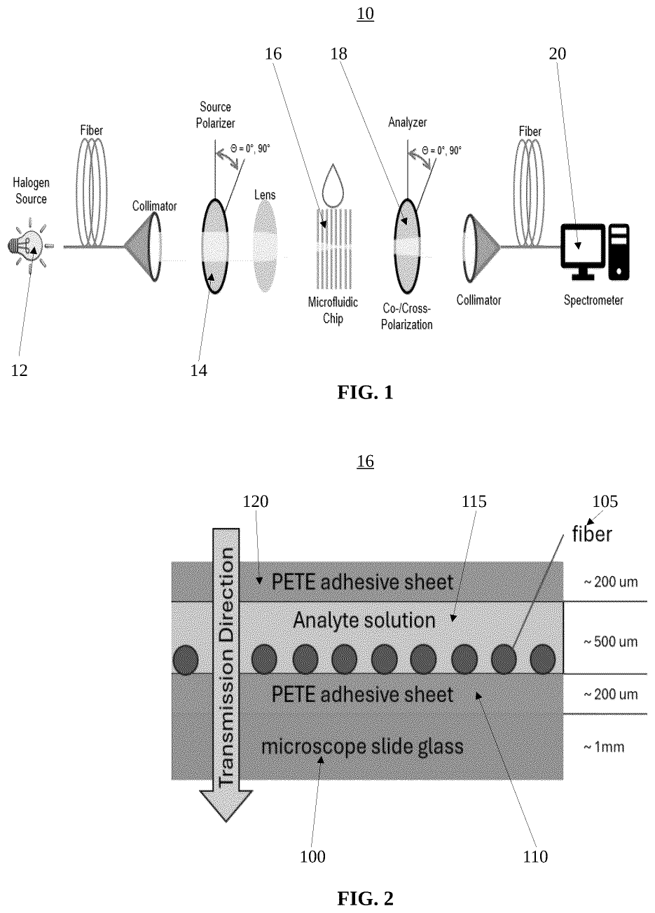

Referring now to FIG. 1, an exemplary optical polarimeter that may be used to detect blood glucose (BG) includes a light source 12, a polarizer 14, an exemplary microfluidic chip 16, an analyzer 18 and a detector 20.

The polarimeter 10 uses the LED source 12, the fixed polarizer 14 and the analyzer 18 to detect changes in rotation of plane-polarized light in the presence of a sample on the microfluidic chip 16. A graph is produced that shows a clear change in the light's polarization with respect to angle. This enables a user to determine various characteristics, including the identity, of the specific chemical compound being investigated.

More specifically, incident non-polarized light is transmitted through the fixed polarizer 14 that only allows a certain orientation of the electric field (E-field) of the light into the sample on the microfluidic chip 16. The sample on the microfluidic chip 16 then rotates the light at a unique angle. As the analyzer 18 is turned, the rotated E-field of the light is maximally transmitted at that unique angle, enabling the user to determine properties of the sample. A (+) enantiomer rotates the plane of linearly polarized light clockwise, dextro, as seen by the detector 20. A (−) enantiomer rotates the plane counter-clockwise, levo.

Turning to FIG. 2, the exemplary microfluidic chip 16, in one embodiment, includes a transparent substrate 100, and a sample array of microfibers 105 mounted on a first double-sided adhesive plastic sheet 110 adhered to the transparent substrate 100 using a custom fiber winder such that the microfibers are facing up and away from the transparent substrate 100. The microfluidic chip 16 also includes a shim 115 added onto the first double-sided adhesive plastic sheet 110 adjacent to the sample array of microfibers 105 such that a microfluidic channel is formed above the sample array of microfibers 105. The microfluidic chip 16 includes a blunt precision tip needle (not shown) added as a fill port to the microfluidic channel. The microfluidic chip 16 includes a second double-sided adhesive plastic sheet 120 laid over the shim 115 to cover the microfluidic channel.

In embodiments, the transparent substrate 100 is a microscope slide glass, which ensures flatness.

In embodiments, the first and second double-sided adhesive plastic sheets 110, 120 include a polyethylene terephthalate (PETE) backing with an acrylic-based adhesive.

In embodiments, the sample array of microfibers 105 includes a ferromagnetic alloy core (e.g. CoFeSiB) and an electrically insulating dielectric (e.g. borosilicate glass) shell microfibers. In one example, the microfibers have a core of 20 μm and an outer diameter of 27 μm. In one example, the shim 115 is 500-μm.

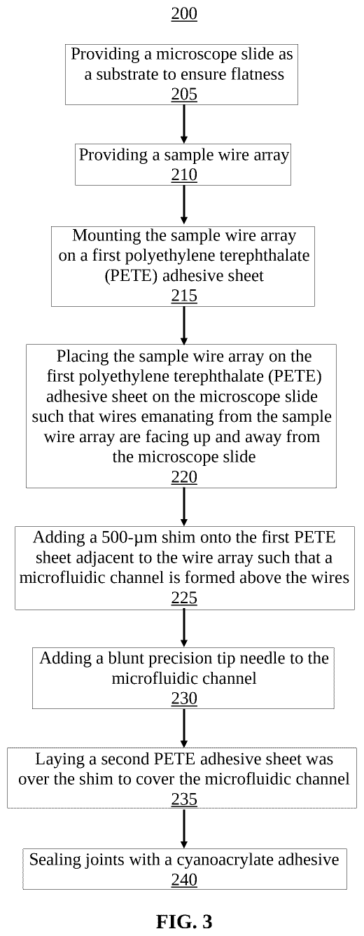

In FIG. 3, an exemplary process 200 of preparing a microfluidic chip includes providing (205) a microscope slide as a substrate for ease of handling and a microscopic flatness.

Process 200 provides (210) a sample wire array and mounting (215) the sample wire array on a first adhesive sheet (e.g., polyethylene terephthalate, PETE).

Process 200 places (220) the sample wire array on the first adhesive sheet on the microscope slide such that wires emanating from the sample wire array are facing up and away from the microscope slide.

Process 200 may also include adding (225) a 500-μm shim onto the first adhesive (e.g. PETE) sheet adjacent to the wire array such that a microfluidic channel is formed above the wires and in-between the wires.

Process 200 may also include adding (230) a blunt precision tip needle to the microfluidic channel, laying (235) a second adhesive sheet was over the shim to cover the microfluidic channel, and sealing (240) joints with a cyanoacrylate adhesive.

In summary, blood sugar is an important biomarker in the diagnosis and management of diabetes mellitus. Current sensing methods are invasive, painful, intermittent, and require complex chemical enzyme interactions. An alternative minimally invasive optical sensing method is presented herein. The invention described above is a sub-mm thick transmission optical microfluidic chip utilizing a ferromagnetic core/dielectric shell microfiber array grating that is used for polarimetric sensing of an optically active solution. In one example, the tested concentration range was from 0.01 mg/mL to 100.0 mg/mL, which includes the concentration range found in human sweat for glucose. A 50-μm microfiber array microfluidic chip was found to magnify the optical rotation by the sucrose analyte solution by several orders of magnitude within the tested range, over a broad spectral band of a linearly polarized light source with the electrical field oriented orthogonal to the microfibers. A calibration curve of the optical rotation was 0.1428 and 0.1225 degrees per logarithmic concentration (mg/mL) for wavelengths of 1000 and 1200 nm, respectively.

Due to the flexibility and scalability of the microfiber and their arrays, the wicking effect of the array and its capability for iontophoresis for sweat generation and extraction, as well as measurement range that covers the concentrations found in sweat, the present invention has the potential for enabling many applications, one of which is a wearable sweat sensor of glucose.

It would be appreciated by those skilled in the art that various changes, such as the microfiber core and/or shell, and modifications, such as the substrate and adhesion, can be made to the illustrated embodiments without departing from the spirit of the present invention. All such modifications and changes are intended to be within the scope of the present invention except as limited by the scope of the appended claims.

What is claimed is:

1. A microfluidic chip comprising:
a transparent or reflective substrate;
a sample array of microfibers mounted on a first double- sided adhesive sheet adhered to the substrate such that the microfibers are facing up and away from the substrate;
a shim added onto the first double-sided adhesive sheet adjacent to the sample array of microfibers such that a microfluidic channel is formed above the sample array of microfibers;
a fluidic inlet added as a fill port to the microfluidic channel; and
a second double-sided adhesive sheet laid over the shim to cover the microfluidic channel.

2. The microfluidic chip of claim 1 wherein the substrate is a microscope slide glass.

3. The microfluidic chip of claim 2 wherein the first and second double-sided adhesive sheets comprise a polyethylene terephthalate (PETE) backing with an acrylic-based adhesive.

4. The microfluidic chip of claim 3 wherein the sample array of microfibers comprises a ferromagnetic alloy core and dielectric shell microfibers.

5. The microfluidic chip of claim 4 wherein the microfibers have a core of 1 to 100 μm and an outer diameter of 2 to 110 μm.

6. The microfluidic chip of claim 5 wherein the shim is 0.1 to 1 mm.

7. An optical polarimeter comprising:
a light source;
a polarizer;
a microfluidic chip;
an analyzer; and
a detector.

8. The optical polarimeter of claim 7 wherein the light source is a halogen source or a white light source.

9. The optical polarimeter of claim 8 further comprising a first collimator positioned between the light source and the polarizer.

10. The optical polarimeter of claim 9 further comprising a lens positioned between the polarizer and the microfluidic chip.

11. The optical polarimeter of claim 10 further comprising a second collimator positioned between the analyzer and the detector.

12. The optical polarimeter of claim 7 wherein the microfluidic chip comprises:
a transparent or reflective substrate;
a sample array of microfibers mounted on a first double-sided adhesive sheet adhered to the substrate using a customfiber winder such that they are facing up and away from the transparent substrate;
a shim added onto the first double-sided adhesive sheet adjacent to the sample array of microfibers such that a microfluidic channel is formed above the sample array of microfibers;
a fill port to the microfluidic channel; and
a second double-sided adhesive plastic sheet laid over the shim to cover the microfluidic channel.

13. The optical polarimeter of claim 12 wherein the substrate is a microscope slide glass.

14. The optical polarimeter of claim 13 wherein the first and second double-sided adhesive plastic sheets comprise a polyethylene terephthalate (PETE) backing with an acrylic-based adhesive.

15. The optical polarimeter of claim 14 wherein the sample array of microfibers comprises a ferromagnetic alloy core and dielectric shell microfibers.

16. The optical polarimeter of claim 15 wherein the microfibers have a core of 1 to 100 μm and an outer diameter of 2 to 110 μm.

17. The microfluidic chip of claim 16 wherein the shim is 0.1 to 1 mm.

18. A method of fabricating a microfluidic chip comprising:
providing a microscope slide as a substrate to ensure local or microscopic flatness;
providing a sample wire array;
mounting the sample wire array on a first adhesive sheet; and
placing the sample wire array on the first adhesive sheet on the microscope slide such that wires emanating from the sample wire array are facing up and away from the microscope slide.

19. The method of claim 18 further comprising adding a shim onto the first sheet adjacent to the wire array such that a microfluidic channel is formed above the wires.

20. The method of claim 19 further comprising:
adding a blunt needle to the microfluidic channel;
laying a second adhesive sheet was over the shim to cover the microfluidic channel; and
sealing joints with a adhesive.
